(19) United States
(12) Patent Application Publication
(10) Pub. No.: US 2015/0201974 A1
(43) Pub. Date: Jul. 23, 2015

(54) SURGICAL INSTRUMENT AND METHOD OF USE

(71) Applicant: WARSAW ORTHOPEDIC, INC., Warsaw, IN (US)

(72) Inventors: Steven D. DeRidder, Bartlett, TN (US); Trevor Seck, Memphis, TN (US)

(73) Assignee: WARSAW ORTHOPEDIC, INC., Warsaw, IN (US)

(21) Appl. No.: 14/160,195

(22) Filed: Jan. 21, 2014

(51) Int. Cl. A61B 17/70

(52) U.S. Cl. CPC A61B 17/7074

(57) ABSTRACT

A surgical instrument system comprises a first member and a second member. The second member is movable relative to the first member. A sensor is configured to measure a distance between the members and digital indicia representing at least one data element of a spinal implant is selected based on the distance. Systems and methods are disclosed.

### TECHNICAL FIELD

The present disclosure generally relates to surgical implants for the treatment of spinal disorders, and more particularly to a surgical instrument and method for treatment of a spine disorder.

### BACKGROUND

Spinal disorders such as degenerative disc disease, disc herniation, osteoporosis, spondylolisthesis, stenosis, scoliosis and other curvature abnormalities, kyphosis, tumor, and fracture may result from factors including trauma, disease and degenerative conditions caused by injury and aging. Spinal disorders typically result in symptoms including pain, nerve damage, and partial or complete loss of mobility.

Non-surgical treatments, such as medication, rehabilitation and exercise can be effective, however, may fail to relieve the symptoms associated with these disorders. Surgical treatment of these spinal disorders includes discectomy, laminectomy, fusion and implantable prosthetics. As part of these surgical treatments, implants, such as, for example, spinal constructs including plates, rods, and fasteners, and interbody devices are often employed for stabilization of a treated section of a spine. During such surgical treatments, surgical instruments may be used to facilitate delivery and placement of the implants. This disclosure describes an improvement over these prior art technologies.

### SUMMARY

In one embodiment, a surgical instrument is provided. The surgical instrument comprises a first member. A second member is movable relative to the first member. A sensor is configured to measure a distance between the members. Digital indicia represents at least one data element of a spinal implant selected based on the distance. In some embodiments, systems and methods are provided.

### BRIEF DESCRIPTION OF THE DRAWINGS

The present disclosure will become more readily apparent from the specific description accompanied by the following drawings, in which:

FIG. 1 is a perspective view of components of one embodiment of a surgical instrument of a surgical system in accordance with the principles of the present disclosure;

FIG. 2 is a top view of the components shown in FIG. 1;

FIG. 3 is a plan view of the components shown in FIG. 1 disposed with vertebrae;

FIG. 4 is a plan view of components of a surgical implant disposed with vertebrae;

FIG. 5 is a plan view of components of one embodiment of a surgical instrument of a surgical system in accordance with the present disclosure disposed with vertebrae;

FIG. 6 is a plan view of components, in part phantom, of one embodiment of a surgical instrument of a surgical system in accordance with the present disclosure disposed with vertebrae; and

FIG. 7 is a plan view of components, in part phantom, of one embodiment of a surgical instrument of a surgical system in accordance with the present disclosure disposed with vertebrae.

### DETAILED DESCRIPTION

The exemplary embodiments of the surgical system and related methods of use disclosed are discussed in terms of medical devices for the treatment of musculoskeletal disorders and more particularly, in terms of a surgical instrument and method for treatment of a spine disorder.

In some embodiments, the presently disclosed system includes one or more instruments having components that are employed with a method for selecting spinal implants. In some embodiments, the selection can include configuration and/or dimension of the implants and/or can be based on factors, which may include, size, distance and/or condition of all or only a portion of an anatomy, such as, for example, a portion of a spine, which may include selected vertebral levels. In some embodiments, the implant can include a spinal plate that is selected based on factors of a spine that can be measured by an instrument of the present system resulting in selection of the plate based on, such as, for example, the overall length of the plate or the center-to-center distance between the outermost screw holes. In one embodiment, the instrument includes scaled calipers that facilitate plate selection.

In one embodiment, the present system comprises a surgical instrument including a caliper for use with one or more spinal plate systems and includes a digital readout. In one embodiment, the surgical instrument includes a programmable electronic display having one or more selectable inputs. In some embodiments, the surgical instrument includes a first selectable input to select a particular plate system for use with vertebrae. In one embodiment, the surgical instrument includes a second selectable input to select whether a read-out will correspond to overall plate length or to center-to-center distance of outermost screw holes of a plate. In one embodiment, the programmable electronic display may be modified for one or more different and/or additional spinal constructs and/or implant systems.

In one embodiment, the surgical instrument utilizes a programmable electronic display that contains one selectable input. In one embodiment, the surgical instrument is configured as a universal spinal rod measuring device. In one embodiment, the instrument has universal tips to accommodate one or more alternate screw extenders.

In one embodiment, one or all of the components of the system are disposable, peel-pack, pre-packed sterile devices used with an implant. One or all of the components of the system may be reusable. The system may be configured as a kit with multiple sized and configured components.

The present disclosure may be understood more readily by reference to the following detailed description of the disclosure taken in connection with the accompanying drawing figures, which form a part of this disclosure. It is to be understood that this disclosure is not limited to the specific devices, methods, conditions or parameters described and/or shown herein, and that the terminology used herein is for the purpose of describing particular embodiments by way of example only and is not intended to be limiting of the claimed disclosure. Also, in some embodiments, as used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment. It is also understood that all spatial references, such as, for example, horizontal, vertical, top, upper, lower, bottom, left and right, are for illustrative purposes only and can be varied within the scope of the disclosure. For example, the references “upper” and “lower” are relative and used only in the context to the other, and are not necessarily “superior” and “inferior”.

Further, as used in the specification and including the appended claims, “treating” or “treatment” of a disease or condition refers to performing a procedure that may include administering one or more drugs to a patient (human, normal or otherwise or other mammal), in an effort to alleviate signs or symptoms of the disease or condition. Alleviation can occur prior to signs or symptoms of the disease or condition appearing, as well as after their appearance. Thus, treating or treatment includes preventing or prevention of disease or undesirable condition (e.g., preventing the disease from occurring in a patient, who may be predisposed to the disease but has not yet been diagnosed as having it). In addition, treating or treatment does not require complete alleviation of signs or symptoms, does not require a cure, and specifically includes procedures that have only a marginal effect on the patient. Treatment can include inhibiting the disease, e.g., arresting its development, or relieving the disease, e.g., causing regression of the disease. For example, treatment can include reducing acute or chronic inflammation; alleviating pain and mitigating and inducing re-growth of new ligament, bone and other tissues; as an adjunct in surgery; and/or any repair procedure. Also, as used in the specification and including the appended claims, the term “tissue” includes soft tissue, vessels, ligaments, tendons, cartilage and/or bone unless specifically referred to otherwise.

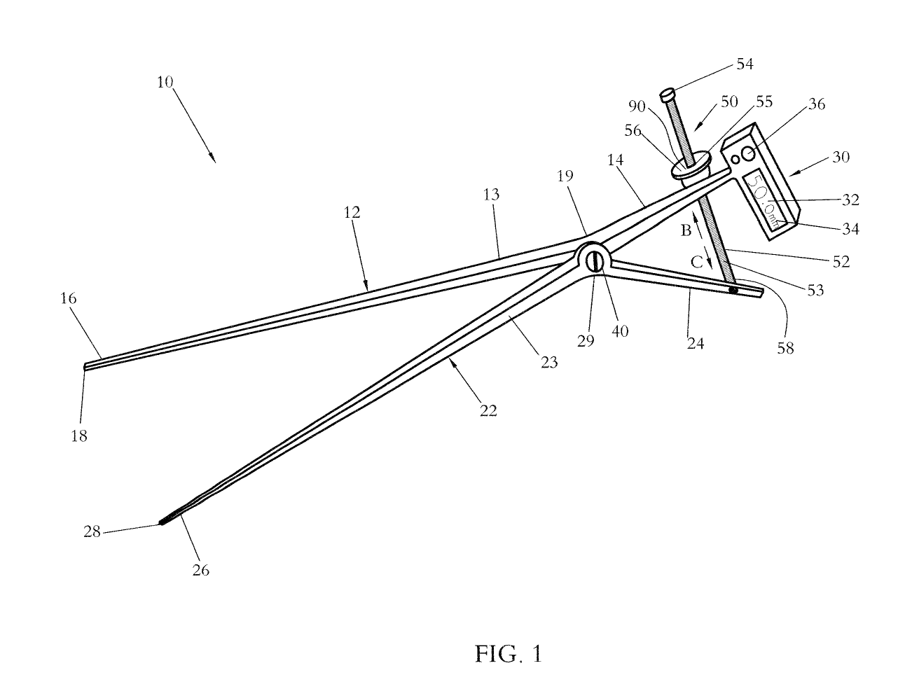

The following discussion includes a description of a system including spinal implants and surgical instruments, related components and methods of employing the system in accordance with the principles of the present disclosure. Alternate embodiments are also disclosed. Reference is made in detail to the exemplary embodiments of the present disclosure, which are illustrated in the accompanying figures. Turning to FIGS. 1-3, there are illustrated components of a surgical system including a surgical instrument 10 in accordance with the principles of the present disclosure.

The components of the surgical system including surgical instrument 10 can be fabricated from biologically acceptable materials suitable for medical applications, including metals, synthetic polymers, ceramics, bone material, tissue and/or their composites. For example, the components of surgical instrument 10, individually or collectively, can be fabricated from materials such as stainless steel alloys, aluminum, commercially pure titanium, titanium alloys, Grade 5 titanium, super-elastic titanium alloys, cobalt-chrome alloys, stainless steel alloys, superelastic metallic alloys (e.g., Nitinol, super elasto-plastic metals, such as GUM METAL® manufactured by Toyota Material Incorporated of Japan), ceramics and composites thereof, thermoplastics such as polyaryletherketone (PAEK) including polyetheretherketone (PEEK), polyetherketoneketone (PEKK) and polyetherketone (PEK), carbon-PEEK composites, PEEK-BaSO₄ polymeric rubbers, polyethylene terephthalate (PET), fabric, silicone, polyurethane, silicone-polyurethane copolymers, polymeric rubbers, polyolefin rubbers, hydrogels, semi-rigid and rigid materials, elastomers, rubbers, thermoplastic elastomers, thermoset elastomers, elastomeric composites, rigid polymers including polyphenylene, polyamide, polyimide, polyetherimide, polyethylene, epoxy, totally resorbable materials, and their combinations. Various components of surgical instrument 10 may have material composites, including the above materials, to achieve various desired characteristics such as strength, rigidity, elasticity, compliance, biomechanical performance, durability and radiolucency or imaging preference. The components of surgical instrument 10, individually or collectively, may also be fabricated from a heterogeneous material such as a combination of two or more of the above-described materials. The components of surgical instrument 10 may be monolithically formed, integrally connected or include fastening elements and/or instruments, as described herein.

Surgical instrument 10 includes a member, such as, for example, an arm 12 and a member, such as, for example, an arm 22 movable relative to arm 12. Arm 12 extends between a proximal end 14 and a distal end 16. End 16 is configured for disposal adjacent a surgical site including spinal tissue. End 16 includes a tip 18. Tip 18 may have various cross-sectional geometries, such as, for example oval, oblong, triangular, square, polygonal, irregular, uniform, non-uniform, offset, staggered, undulating, arcuate, variable and/or tapered. In one embodiment, tip 18 has a sharpened configuration for precise measurement, as described herein. In one embodiment, tip 18 is blunt or rounded to avoid damage to tissue. End 14 includes an outer gripping surface 13 configured to facilitate gripping by a practitioner. Gripping surface 13 may be, for example, rough, arcuate, undulating, mesh, porous, semi-porous, dimpled and/or textured.

Arm 22 includes a proximal end 24 and a distal end 26. Arm 22 is moveable relative to arm 12, as discussed herein. End 26 is configured for disposal adjacent a surgical site. End 26 includes a tip 28. Tip 28 may have various cross-sectional geometries, such as, for example oval, oblong, triangular, square, polygonal, irregular, uniform, non-uniform, offset, staggered, undulating, arcuate, variable and/or tapered. In one embodiment, tip 28 has a sharpened configuration for precise measurements, as described herein. In one embodiment, tip 28 is blunt or rounded to avoid damage to tissue. End 24 includes an outer gripping surface 23 configured to facilitate gripping by a practitioner. Gripping surface 23 may be, for example, rough, arcuate, undulating, mesh, porous, semi-porous, dimpled and/or textured.

Arms 12, 22 are joined by a connector such as, for example a pivot hinge 40 comprising a screw or pin movably fixed with arms 12, 22. Hinge 40 is located adjacent ends 14, 24 of arms 12, 22. In one embodiment, hinge 40 is disposed adjacent to ends 14, 24 such that ends 16, 26 are longer than ends 14, 24. In one embodiment, hinge 40 is disposed at a midpoint of arms 12, 22, such that ends 14, 24 are of equal length. Arms 12, 22 each include an inner surface that defines an opening configured for disposal of pivot hinge 40. The openings are aligned to receive pivot hinge 40 and facilitate relative rotation of arms 12, 22. In some embodiments, the connector may be a pin, bolt, rivet, latch, clamp, clasp, hook, fastener, or clip. Arm 22 rotates relative to arm 12 about a central axis of hinge 40.

Surgical instrument 10 includes an actuator, such as, for example, a drive mechanism 50 disposed with arms 12, 22. Drive mechanism 50 includes a shaft 52 and a translation part, such as, for example, a nut 56 defining a threaded cavity. Shaft 52 extends between ends 14, 24. Shaft 52 includes a threaded outer surface 53 configured to engage arms 12, 22. Shaft 52 comprises a stop, such as, for example, a head 54 having a diameter greater than shaft 52. Head 54 is configured as a stop limit defining a limit for movement of arm 22 relative to arm 12. An end 58 of shaft 52 is fixedly connected to arm 22 to facilitate movement of arm 22 relative to arm 12.

Nut 56 includes a central channel 55 configured for rotational engagement with shaft 52. Nut 56 rotates in a clockwise and counter-clockwise direction for axial translation of nut 56 in the directions shown by arrows B and C in FIG. 1, along shaft 52 causing end 14 to translate along shaft 52 towards and away from end 24. Such translation causes end 16 to translate relative to end 26. Nut 56 includes a threaded surface of channel 55 configured to engage surface 53 of shaft 52 such that rotation of nut 56 axially translates shaft 52 relative to nut 56.

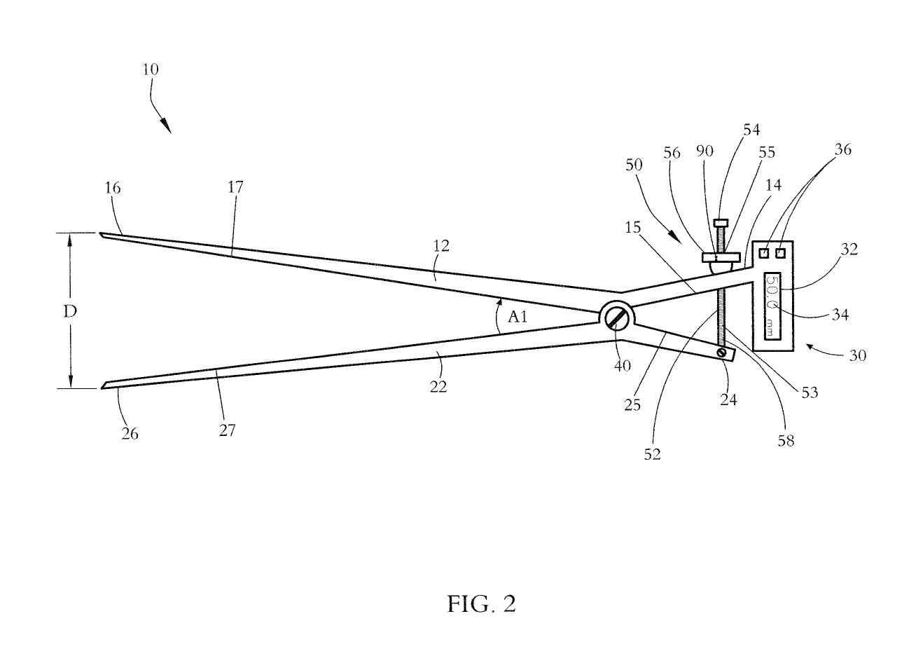

Rotating nut 56 in a clockwise direction causes translation of nut 56, in the direction shown by arrow C, relative to shaft 52. Rotation of nut 56, in the direction shown by arrow C, causes distal ends 16, 26 to translate such that angle A1 and distance D, as shown in FIG. 2, increase to measure a factor, such as, for example, a linear distance of a portion of a spine anatomy for selecting and determining configuration and/or dimension of a selected implant for disposal with the spine anatomy.

Rotating nut 56 in a counter-clockwise direction causes translation of nut 56, in the direction shown by arrow B, relative to shaft 52. Rotation of nut 56, in the direction shown by arrow B, causes distal ends 16, 26 to translate such that angle A1 and distance D, as shown in FIG. 2, decrease to measure a factor, such as, for example, a linear distance of a portion of a spine anatomy for selecting and determining configuration and/or dimension of a selected implant for disposal with the spine anatomy.

Surgical instrument 10 includes a sensor 90 disposed with a cavity of end 14. Sensor 90 is disposed for engagement with nut 56 and shaft 52. Sensor 90 senses translation of nut 56 relative to shaft 52 corresponding to angle A1 and/or distance D, as described herein, and includes a transducer in communication with a display module and processor, as described herein, to display anatomical information and/or provide implant selection information, as measured by arms 12, 22 and tips 18, 28. In some embodiments, sensor 90 senses rotation of nut 56 relative to shaft 52 in rotational units corresponding to angle A1 and/or distance D as measured by arms 12, 22 and tips 18, 28. In some embodiments, sensor 90 senses a linear distance traveled by nut 56 relative to shaft 52 in axial translation corresponding to angle A1 and/or distance D as measured by arms 12, 22 and tips 18,28.

In one embodiment, sensor 90 can be disposed within arm 12, arm 22, hinge 40 or both arms 12, 22. In one embodiment, sensor 90 is disposed adjacent hinge 40 such that as arm 22 is rotated away from arm 12 from a first orientation to a second orientation, sensor 90 reads the angular variance between the first orientation and the second orientation. Surgical instrument 10 may include a sensor, such as, for example, one or more accelerometers, rotary capacitive sensors, a solid-state sensor incorporating an accelerometer or a potentiometer, solid-state sensors employing other physical properties (e.g., a magnetic field sensor or other device that employs magnetic resistance), angular position sensors, rotary position sensors, linear position sensors, mechanical or electromagnetic induction sensors, capacitive sensors and/or gate sensors.

Surgical instrument 10 includes a display module 30, which includes a processor, such as, for example, a microcontroller (not shown). Display module 30 is configured to represent one or more data elements of a spinal implant, a spinal anatomy and/or a spinal implant selected based on a measured dimension, such as, for example, a measured distance or angle, as described herein, based on the spinal anatomy. Display module 30 includes a generally rectangular shape. In some embodiments, display module 30 may comprise a variety of other shapes, such as circular, elliptical, square, or polygonal. Display module 30 comprises a screen 32 configured to display digital indicia 34 representing one or more data elements, as described herein. In some embodiments, screen 32 may include cathode ray tube, light-emitting diode display, liquid crystal display, electronic ink, electroluminescent display, or plasma display panel.

Display module 30 includes one or more inputs, such as, for example, buttons 36 as an interface with the processor of module 30 and digital indicia 34. In some embodiments, buttons 36 are configured to provide display and/or alter display information and system settings such as, for example, powering on/off, switching display modes, changing information displayed, displaying battery information, changing display settings such as screen brightness, calibrating the screen, and/or calibrating the sensor.

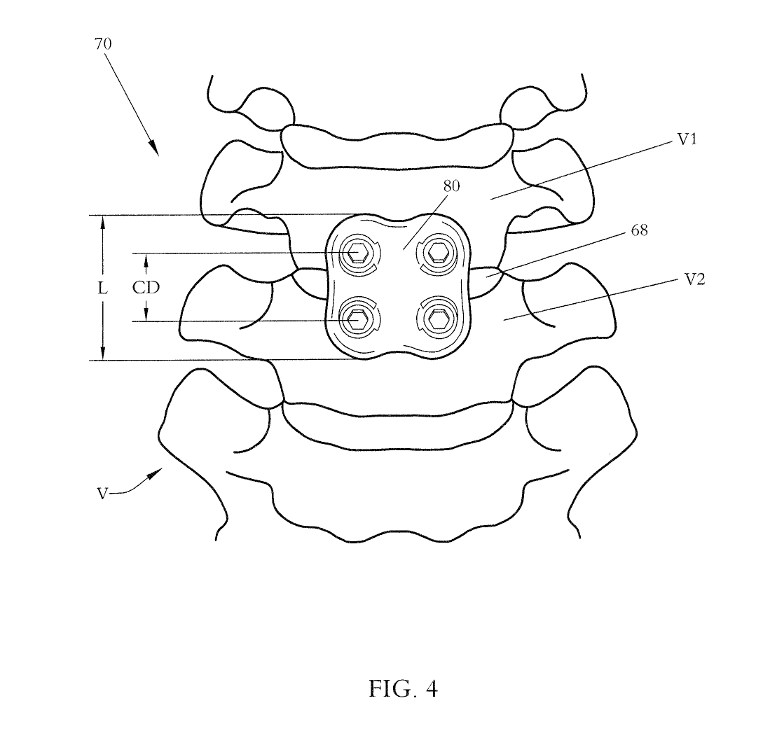

In one embodiment, a first programmable input, such as, for example, one of buttons 36 provides for selection of an implant, implant system, for example, a particular plate system, and a second programmable input, such as, for example, one of buttons 36 determines a display format, such as, for example, overall plate length L, as shown in FIG. 4, or center-to-center distance CD of the outermost screw holes. In some embodiments, a programmable input, such as, for example, a keyboard/computer can interface module 30 via wired or wireless communication.

The microcontroller of module 30 is in communication with sensor 90 and processes the information obtained by sensor 90 and generates digital indicia 34 for display from screen 32. In some embodiments, the measured distance D and/or angle A1 is sensed by sensor 90 and communicated to the microcontroller of module 30. The measured distance D and/or angle A1 of a spinal anatomy is compared with preprogrammed information, such as, for example, dimension and configuration data corresponding to spinal implants, such as, for example, spinal plates and/or spinal plate systems that match with the measured distance D and/or angle A1 of the spinal anatomy. The microcontroller determines and/or selects a spinal plate and/or spinal plate system corresponding to the measured distance D and/or angle A1 of the spinal anatomy and displays pre-programmed and/or selected digital indicia 34 from screen 32.

In some embodiments, the microcontroller may be variously disposed with the components of surgical instrument 10 and connected by circuit to sensor 90. In one embodiment, the microcontroller is programmable to include at least one data element of a spinal implant. In some embodiments, the microcontroller can be programmed with one or a plurality of alternately configured and/or dimensioned spinal implants, such as, for example, one of a plurality of spinal plates. In one embodiment, the microcontroller is programmable to include a plurality of data elements for various spinal implants. In one embodiment, the microcontroller is programmable to include overall length of a spinal implant and/or the distance between a center of a first screw hole and a center of a second screw hole of a spinal plate. In one embodiment, the spinal implant may be a spinal rod, as described herein. In some embodiments, the spinal implant may be a spinal tether.

In assembly, operation and use, a surgical system including surgical instrument 10, similar to the systems described herein, is employed with a surgical procedure for treatment of a spinal disorder affecting a section of a spine of a patient, as discussed herein. The surgical system is employed with a surgical procedure for treatment of a condition or injury of an affected section of the spine including one or more vertebrae V. In some embodiments, surgical instrument 10 is used to measure a selected portion of vertebrae V. This measurement information is used to determine a selected configuration or dimension of a selected spinal implant, such as, for example, spinal plate 80, as shown in FIG. 4.

For example, surgical instrument 10 can be employed with a surgical arthrodesis procedure, such as, for example, an interbody fusion for treatment of an applicable condition or injury of an affected section of vertebrae V and adjacent areas within a body, such as, for example, intervertebral disc space 68 between a vertebra V1 and a vertebra V2 having an anterior side A and a posterior side P. In some embodiments, surgical instrument 10 is used to measure distance between anatomical locations on vertebrae V1, V2 to select and/or determine an implant size and/or configuration for treating vertebrae V.

A medical practitioner obtains access to a surgical site including vertebrae V1, V2 in any appropriate manner, such as through incision and retraction of tissues. Surgical instrument 10 can be used in any existing surgical method or technique including open surgery, mini-open surgery, minimally invasive surgery and percutaneous surgical implantation, whereby vertebrae V1, V2 are accessed through a mini-incision, or sleeve that provides a protected passageway to the area. In one embodiment, the components of surgical instrument 10 are delivered into a surgical pathway to the surgical site along an anterior surgical approach. Once access to the surgical site is obtained, the particular surgical procedure can be performed for treating the spine disorder.

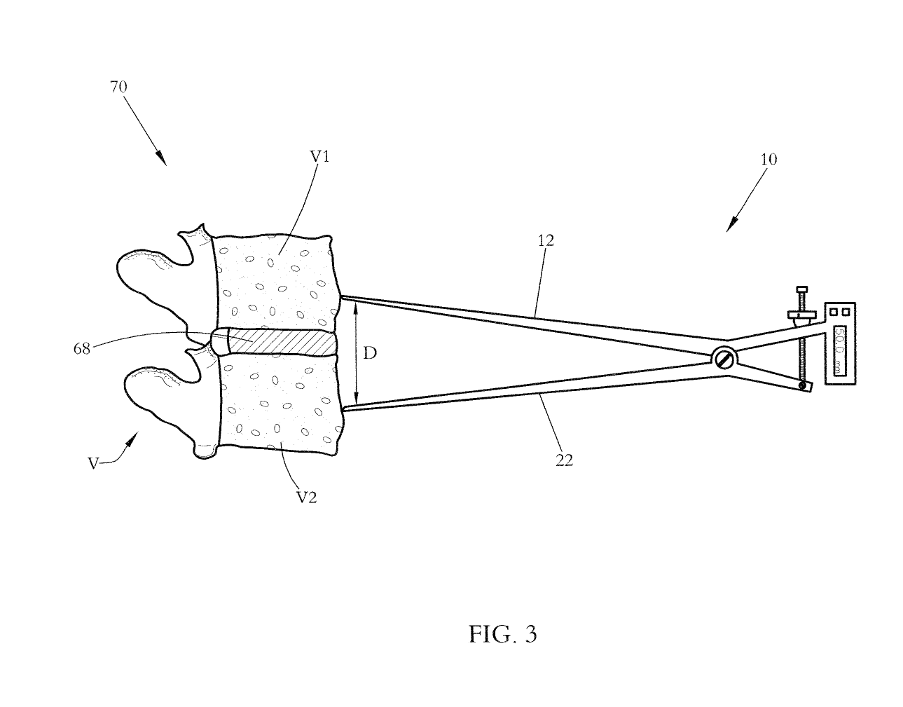

An incision is made in the body of the patient and a cutting instrument (not shown) creates the surgical pathway for insertion of components of the surgical system. Arms 12, 22 of surgical instrument 10 are disposed adjacent to the surgical site and into engagement with vertebrae V1, V2, as shown in FIG. 3, such that tips 18, 28 contact vertebrae V1, V2. In some embodiments, tips 18, 28 do not contact vertebrae V1, V2. A medical practitioner identifies a targeted area or a plurality of areas of vertebrae V1, V2 to be measured for implantation of plate 80. In some embodiments, surgical instrument 10 is utilized in a multi-level application, such as, for example, multiple locations along vertebrae V.

Tips 18, 28 are disposed adjacent vertebrae V1, V2 to measure a distance D along anterior portion A for implantation of plate 80 with vertebrae V. Nut 56 is rotated in a clockwise direction or a counter-clockwise direction to translate nut 56, in the directions shown by arrows B and C in FIG. 3, relative to shaft 52. Translation of nut 56 causes distal ends 16, 26 to rotate to measure angle A1 and distance D, as shown in FIG. 2, for selecting and determining configuration and/or dimension of plate 80.

Sensor 90 senses translation of nut 56 relative to shaft 52 corresponding to the measured angle A1 and/or distance D. The measured distance D and/or angle A1 is sensed by sensor 90 and communicated to the microcontroller of module 30. The microcontroller of module 30 communicates with sensor 90 and processes the information obtained by sensor 90. The measured distance D and/or angle A1 of the selected portion of vertebrae V1, V2 is compared with preprogrammed dimension and configuration data corresponding to a plurality of alternatively dimensioned plates 80 that match with the measured distance D and/or angle A1. The microcontroller determines and/or selects a spinal plate 80 corresponding to the measured distance D and/or angle A1 and displays information corresponding to selected plate 80 as digital indicia 34 from screen 32.

Pilot holes (not shown) are made in vertebrae V1, V2 for receiving bone fasteners in the openings of plate 80. The bone fasteners are inserted, drilled or otherwise fixed to vertebrae V1, V2 to attach plate 80 with vertebrae V. Upon completion of the procedure, surgical instrument 10, other assemblies and non-implanted components of the surgical system are removed and the incision is closed. Surgical instrument 10 can be made of radiolucent materials such as polymers. Radiomarkers may be included for identification under x-ray, fluoroscopy, CT or other imaging techniques. In some embodiments, the use of surgical navigation, microsurgical and image guided technologies may be employed to access, view and repair spinal deterioration or damage, with the aid of surgical instrument 10. In some embodiments, the spinal implant may include one or a plurality of plates, connectors and/or bone fasteners for use with a single vertebral level or a plurality of vertebral levels.

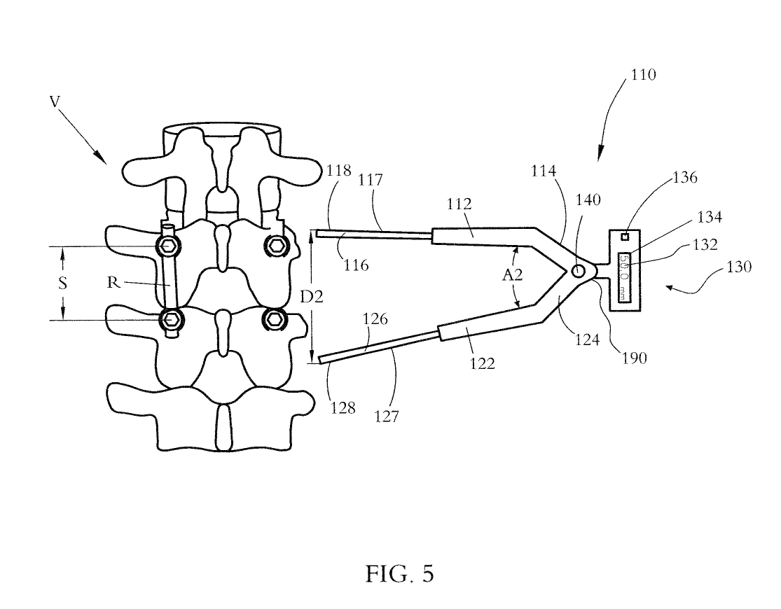

In one embodiment, as shown in FIG. 5, the surgical system, similar to the surgical systems and methods described with regard to FIGS. 1-4, includes a surgical instrument 110 configured for measuring a portion of vertebrae V for selecting and determining configuration and/or dimension of a selected implant, such as, for example, a spinal rod R for disposal with vertebrae V.

Surgical instrument 110 includes a member, such as, for example, an arm 112 and a member, such as, for example, an arm 122 movable relative to arm 112. Arm 112 extends between a proximal end 114 and a distal end 116. End 116 includes an extension 117 and is configured for disposal adjacent a surgical site including spinal tissue. End 116 includes a tip 118, similar to tip 18 described herein. Arm 122 includes a proximal end 124 and a distal end 126. Arm 122 is moveable relative to arm 112. End 126 includes an extension 127 and is configured for disposal adjacent a surgical site. End 126 includes a tip 128, similar to tip 128 described herein.

Arms 112, 122 are joined by a connector such as, for example a pivot hinge 140, similar to hinge 40 described herein, movably fixed with arms 112, 122. Hinge 140 is located adjacent ends 114, 124. Arm 122 rotates relative to arm 112 about a central axis of hinge 140. Arm 122 rotates relative to arm 112 such that an angle A2 and distance D2 measure a factor, such as, for example, a linear distance of a portion of a spine anatomy for selecting and determining configuration and/or dimension of a selected spinal rod R for disposal with vertebrae V.

Surgical instrument 110 includes a sensor 190, similar to sensor 90 described herein, disposed with ends 114, 124. Sensor 190 is disposed for engagement with arms 112, 122. Sensor 190 senses rotation of arm 112 relative to arm 122 corresponding to angle A2 and/or distance D2 and includes a transducer in communication with a display module and processor, as described herein, to display anatomical information and/or provide implant selection information, as measured by arms 112, 122 and tips 118, 128.

Surgical instrument 110 includes a display module 130, similar to module 30 described herein, which includes a processor, such as, for example, a microcontroller (not shown). Display module 130 comprises a screen 132 configured to display digital indicia 134 representing one or more data elements, as described herein. Display module 130 includes a button 136, similar to buttons 36 described herein, as an interface with the processor of module 130 and digital indicia 134.

The microcontroller of module 130 is in communication with sensor 190 and processes the information obtained by sensor 190 and generates digital indicia 134 for display from screen 132. The measured distance D2 and/or angle A2 is sensed by sensor 190 and communicated to the microcontroller of module 130. The measured distance D2 and/or angle A2 of vertebrae V is compared with preprogrammed dimension and configuration data corresponding to spinal rod R that match with the measured distance D2 and/or angle A2 of vertebrae V. The microcontroller determines and/or selects a spinal rod R corresponding to the measured distance D2 and/or angle A2 of vertebrae V and displays pre-programmed and/or selected digital indicia 134 from screen 132.

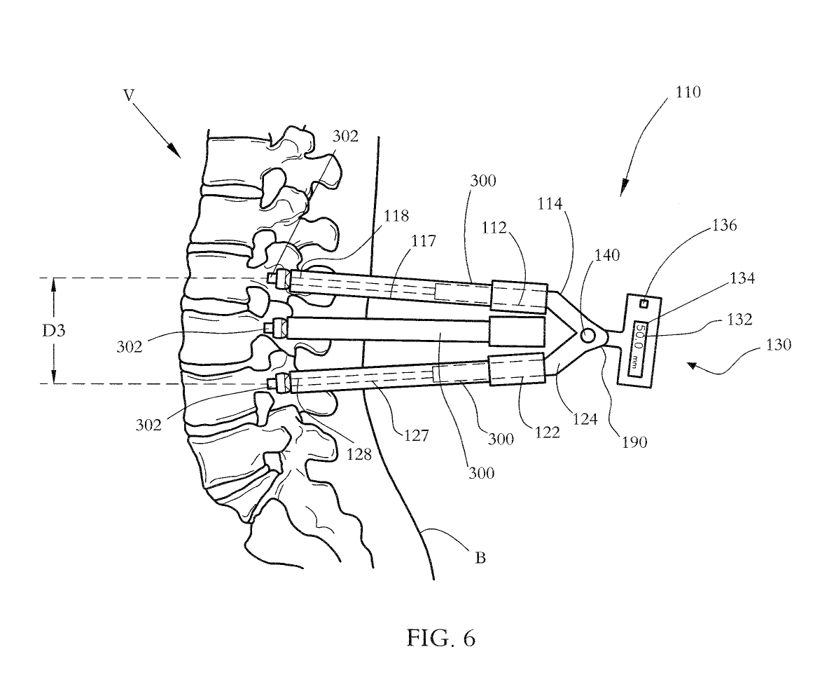

In one embodiment, as shown in FIG. 6, the surgical system, similar to the systems and methods described herein, includes a surgical instrument 110. Surgical instrument 110 is employed for treating a spine, as described herein, which includes vertebrae V using a percutaneous surgical technique. Surgical instrument 110 is employed with the minimally invasive surgery and/or percutaneous surgical implantation, whereby vertebrae V is accessed through a mini-incision, or sleeve that provides a protected passageway to a surgical site.

One or a plurality of incisions are made in a body B of a patient and a cutting instrument (not shown) creates one or a plurality of surgical pathways and/or openings for introduction of surgical instrumentation and/or implantation of components of the surgical system to surgical site, which includes vertebrae V1, V2, V3. In one embodiment, surgical instrument 110 is employed with a percutaneous surgical implantation such that a stab incision creates a surgical pathway for delivering components of the surgical system including surgical instruments, such as, for example, extenders 300 adjacent vertebrae V at the surgical site. In some embodiments, a preparation instrument (not shown) can be employed to prepare tissue surfaces of vertebrae V.

Pilot holes or the like are made in vertebrae V1, V2, V3 for receiving the shafts of pedicle screws 302. Extenders 300 are introduced along each of the surgical pathways corresponding to each of vertebrae V1, V2, V3 and disposed adjacent vertebrae V at the surgical site and the components of the surgical system are manipulable to fix or otherwise connect pedicle screws 302 with vertebrae V. Pedicle screws 302 are fastened with vertebrae V. A driver (not shown) may be employed with extenders 300 to fix pedicle screws 302 with vertebrae V. Extensions 117, 127 and arms 112, 122 are placed into extenders 300 such that tips 118, 128 contact pedicle screws 302. Arms 112, 122 are manipulated to align tips 118, 128 with pedicle screws 302 to obtain a distance D3 between pedicle screws 302 calculated by surgical instrument 110, as described herein.

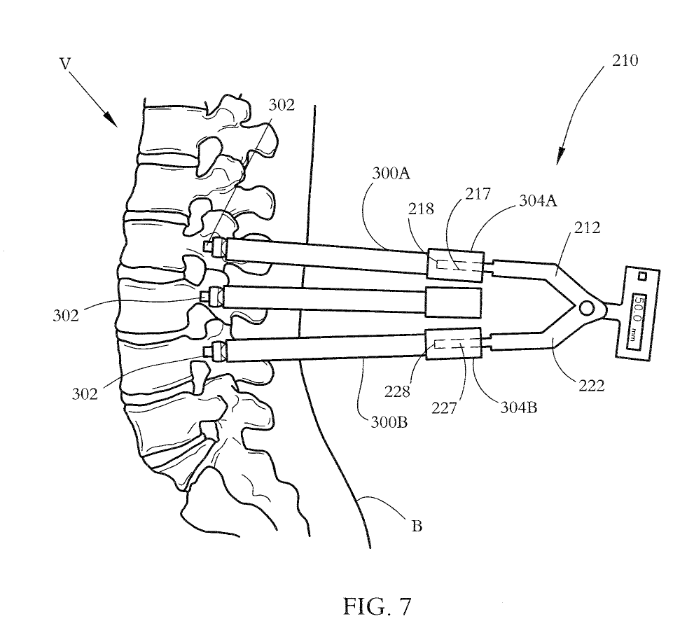

In one embodiment, as shown in FIG. 7, the surgical system, similar to the systems and methods described herein, includes a surgical instrument 210, similar to surgical instrument 110 described with regard to FIGS. 5 and 6. Arms 212, 222 include reduced length extensions 217, 227 having large diameter, spherical tips 218, 228. Tips 218, 228 are placed into extenders 300a, 300b and fixed with proximal ends 304a, 304b of extenders 300a. 300b. Tips 218, 228 engage a flange stop (not shown) of proximal ends 304a, 304b and are disposed in a friction and/or pressure fit with proximal ends 304a. 304b to secure surgical instrument 210 with extenders 300a, 300b. Tips 218, 228 are spaced from and do not contact the heads of pedicle screws 302. As such, the distal end of surgical instrument 210 is engaged with proximal ends 304a, 304b in a configuration such that extenders 300a. 300b are extensions of tips 218, 228. Arms 212, 222 are manipulated to align tips 218, 228 with extenders 300a, 300b connected with pedicle screws 302 to obtain a distance between pedicle screws 302 calculated by surgical instrument 210, as described herein.

It will be understood that various modifications may be made to the embodiments disclosed herein. Therefore, the above description should not be construed as limiting, but merely as exemplification of the various embodiments. Those skilled in the art will envision other modifications within the scope and spirit of the claims appended hereto.

What is claimed is:

1. A surgical instrument comprising:
a first member,
a second member movable relative to the first member;
a sensor being configured to measure a distance between the members; and
digital indicia representing at least one data element of a spinal implant selected based on the distance.

2. A surgical instrument as recited in claim 1, further comprising a microcontroller for processing the distance and generating the indicia.

3. A surgical instrument as recited in claim 2, wherein the microcontroller is programmable to include the at least one data element of the spinal implant.

4. A surgical instrument as recited in claim 2, wherein the microcontroller is programmable to include a plurality of data elements for various spinal implants.

5. A surgical instrument as recited in claim 2, wherein the microcontroller is programmable to include overall length of the implant and the distance between a center of a first screw hole to a center of a second screw hole.

6. A surgical instrument as recited in claim 2, wherein the microcontroller is programmable with data elements of a plurality of alternatively configured spinal implants.

7. A surgical instrument as recited in claim 1, wherein the second member is pivotable relative to the first member.

8. A surgical instrument as recited in claim 1, further comprising a display module including at least one input element.

9. A surgical instrument of claim 1, wherein the spinal implant includes a plate.

10. A surgical instrument as recited in claim 1, wherein the spinal implant includes a rod.

11. A surgical instrument as recited in claim 1, further comprising an actuator configured to rotate the second member relative to the first member.

12. A surgical instrument as recited in claim 11, wherein the sensor reads the actuator to measure the distance.

13. A surgical instrument as recited in claim 1, wherein the first member defines a cavity configured for disposal of the sensor.

14. A surgical instrument comprising:
a first member extending between a first end and a second end;
a second member extending between a first end and a second end, the first ends being connected and the second ends being relatively movable;
a sensor being disposed adjacent to at least one of the first ends and configured to measure a distance between the second ends;
an actuator configured to rotate the second member relative to the first member; and
a module disposed with at least one of the members and including digital indicia of at least one data element of a spinal implant selected based on the distance.

15. A surgical instrument as recited in claim 14, wherein the module further comprises a microcontroller for processing the distance and generating the at least one data element.

16. A surgical instrument as recited in claim 15, wherein the microcontroller is programmable to include a plurality of data elements for various spinal implants.

17. A surgical instrument as recited in claim 15, wherein the microcontroller is programmable to include an overall length of the implant and the distance between a center of a first screw hole to a center of a second screw hole.

18. A surgical instrument as recited in claim 14, wherein the first member defines a cavity configured for disposal of the sensor.

19. A surgical instrument as recited in claim 14, wherein the sensor reads the actuator to measure the distance.

20. A surgical instrument comprising:
a first arm extending between a first end and a second end;
a second arm extending between a first end and a second end, the first ends being connected and the second ends being relatively movable:
a sensor being disposed adjacent to at least one of the first ends and configured to measure a distance between the second ends;
an actuator configured to rotate the second member relative to the first member;
a display module disposed with at least one of the first and second arms and in communication with the sensor, the display module including digital indicia of at least one data element of a spinal implant selected based on the distance, and
a microcontroller for processing the distance and generating the indicia.
